(19) United States
(12) Patent Application Publication
(10) Pub. No.: US 2016/0318674 A1
(43) Pub. Date: Nov. 3, 2016

(54) GAS-TIGHT PHARMACEUTICAL BOTTLE CLOSURE

(71) Applicant: William Eugene LLOYD, Fort Meyers, FL (US)

(72) Inventor: William Eugene LLOYD, Fort Meyers, FL (US)

(21) Appl. No.: 15/205,859

(22) Filed: Jul. 8, 2016

Related U.S. Application Data
(63) Continuation of application No. 14/185,402, filed on Feb. 20, 2014.

(51) Int. Cl. B65D 51/18; A61K 31/198; A61J 1/03; B65D 50/00; B65D 41/04

(52) U.S. Cl. CPC B65D 51/18; B65D 50/00; B65D 41/0442; A61J 1/03; A61K 31/198

(57) ABSTRACT

A gas-tight pharmaceutical container includes a pharmaceutical bottle defining a main enclosure and an upper neck forming a top opening. The upper neck includes an upper rim forming a planar engagement surface. A screw-on closure cap seals the top opening, and includes a circular top member and a circumferential flange. In one embodiment, the closure cap is metal and includes an annular pliable gasket secured to a peripheral portion thereof and a layer of lacquer applied interiorly of the gasket. In another embodiment, the closure cap is plastic and includes a circular inner liner formed by a base layer, a foil layer adjacent to the base layer, and an annular pliable gasket secured to the foil layer. With either embodiment, threaded engagement of the screw-on closure cap with the pharmaceutical bottle under pressure causes the pliable gasket to sealingly engage the upper rim to provide a gas-tight seal.

### CROSS-REFERENCE TO RELATED APPLICATION

This application is a Continuation of U.S. patent application Ser. No. 14/185,402, filed on Feb. 20, 2014, herein incorporated by reference.

### BACKGROUND OF THE INVENTION

Various products are provided in sealed containers in order to preserve the products in the container and prevent degradation. Pharmaceuticals, especially pharmaceuticals which are oxygen-sensitive or highly oxygen-sensitive, such as ultra-oxygen-sensitive levothyroxine sodium tablets, capsules or the like, are one type of product where a good and reliable seal at the open top of the pharmaceutical bottle is important to prevent oxygen in the atmosphere as well as moisture from any source from coming in contact with the pharmaceuticals in the bottle and unnecessarily degrading the pharmaceuticals. For that reason, pharmaceutical containers often include an oxygen scavenger therein, in addition (typically) to a foil seal attached by induction heat sealing to the open top of the bottle and underneath a removable cap provided for the bottle. This foil seal is used after manufacture of the filled bottle, during shipping and prior to opening by the user, at which time the foil seal is removed or destroyed. However, the provision of such a foil seal has proven to be difficult and complex, posing more problems than the closing of the containers themselves.

In the prior art, a very common foil seal for a pharmaceutical bottle at the open top thereof is a heat-sealed foil laminate thermo-adhered to the upper rim or finish of the top of the pharmaceutical bottle. Such a foil laminate is typically used together with a) a metal cap having a pulp liner and a layer of foil adhered to the pulp liner, or b) a plastic cap with a foil laminate liner under a thermo-plastic coating of the cap top. However, the effectiveness of such thermo-adhered foil laminates to the open top of the bottle is dependent on the seal between the bottle and the thermo-adhered foil laminate since heat-sealing (especially polyethylene) bottles, even when done properly, is a difficult operation. This heat-sealing of the thermo-adhered foil laminate to the open top is difficult or undesirable because:
the heat-sealing equipment is complex, and dependent on temperature and dwell time;
the heat-sealed bottles typically need a “re-torqueing” operation after the sealed containers and applied caps have cooled;
the heat-sealing process itself exposes the contents to thermo-degradation and possibly detrimental degradation, such as from the glue fumes;
the heat-sealing is difficult or ineffective if the tops or finishes of the bottles have irregularities (probably the main cause of failure before opening by the user);
the removal of the thermo-adhered foil laminate from the tops of the bottles is an irritation to users; and
after removal (or more typically partial removal) of the thermo-adhered foil laminate, re-sealing is practically impossible with the remaining top (having only a pulp/foil liner or foil laminate) and the uneven surface of the open top of the bottle.

Thus, there is a need in the art for an alternative to the thermo-adhered foil laminate for sealing and resealing of a pharmaceutical bottle or the like.

### BRIEF SUMMARY OF THE INVENTION

Thus, in accordance with the present invention, a gas-tight pharmaceutical container is provided including a pharmaceutical bottle defining a main enclosure and an upper neck extending from the main enclosure and forming a top opening for the main enclosure as well as having external screw threads thereabout. The upper neck also includes an upper rim forming a planar engagement surface about the top opening. The pharmaceutical container also includes a metal screw-on closure cap used to seal the top opening of the bottle. This closure cap includes a circular top member and a circumferential flange depending downwardly from the top member and defining an interior area of the top member. The flange has internal screw threads which mate with the external screws of the upper neck to secure the closure cap under pressure to the planar engagement surface of the upper neck of the main enclosure. The closure cap also includes an annular pliable gasket secured to a peripheral portion of the interior area of the top member and a layer of lacquer applied to the interior area of the top member inside of the gasket. With this construction, threaded engagement of the screw-on closure cap with the pharmaceutical bottle under pressure causes the pliable gasket to sealingly engage the upper rim of the pharmaceutical bottle to provide a gas-tight seal.

The material of the closure cap is preferably either aluminum or steel, while the pharmaceutical bottle is preferably a plastics material. The material of the pliable gasket is preferably one of butyl, silicone, nitrile or fluoroelastomer.

Also in accordance with the present invention a second embodiment of a gas-tight pharmaceutical container is provided including a pharmaceutical bottle as described above. The pharmaceutical container also includes a screw-on closure cap much as noted above, except that this second embodiment includes a circular inner liner which extends over the interior area of the top member of the closure cap. This inner liner includes a base layer which is secured to the interior area of the top member at the top of the flange, a foil layer adjacent to the base layer, and an annular pliable gasket secured to a peripheral area of the foil layer. Then, as with the first-mentioned pharmaceutical container, threaded engagement of the screw-on closure cap with the pharmaceutical bottle under pressure causes the pliable gasket to sealingly engage the upper rim of the pharmaceutical bottle to provide a gas-tight seal.

In this second embodiment, the foil layer is preferably adhered to the base layer. In addition, the material of the pliable gasket is preferably one of butyl, silicone, nitrile or fluoroelastomer. Also preferably, the material of the pharmaceutical bottle is a plastics material and the material of the closure cap is a plastics material.

It is thus a feature of the present invention that an effective seal is provided between a cap and bottle which seals the bottle after manufacture and before use by the user, as well as after the first use and each subsequent use by the user.

It is also a feature of the present invention that an effective seal is provided which is simple and reliable, as well as being effectively resealable after each use.

Other features and advantages of the present invention are stated in or apparent from detailed descriptions of presently preferred embodiments of the invention found hereinbelow.

### BRIEF DESCRIPTION OF THE SEVERAL VIEWS OF THE DRAWINGS

FIG. 1 is a cross sectional elevation view of a top portion of a gas-tight pharmaceutical bottle of the present invention including a closure cap.

FIG. 2 is a bottom view of the closure cap for the gas-tight pharmaceutical bottle depicted in FIG. 1.

FIG. 3 is cross-sectional elevation view of a portion of the closure cap identified by the broken line 3 in FIG. 1.

FIG. 4 is a cross sectional elevation view of a top portion of a second embodiment of a gas-tight pharmaceutical bottle of the present invention including another closure cap.

FIG. 5 is a bottom view of the closure cap for a gas-tight pharmaceutical bottle depicted in FIG. 4.

FIG. 6 is cross-sectional elevation view similar to FIG. 3 but of a portion of still another closure cap.

### DETAILED DESCRIPTION OF THE INVENTION

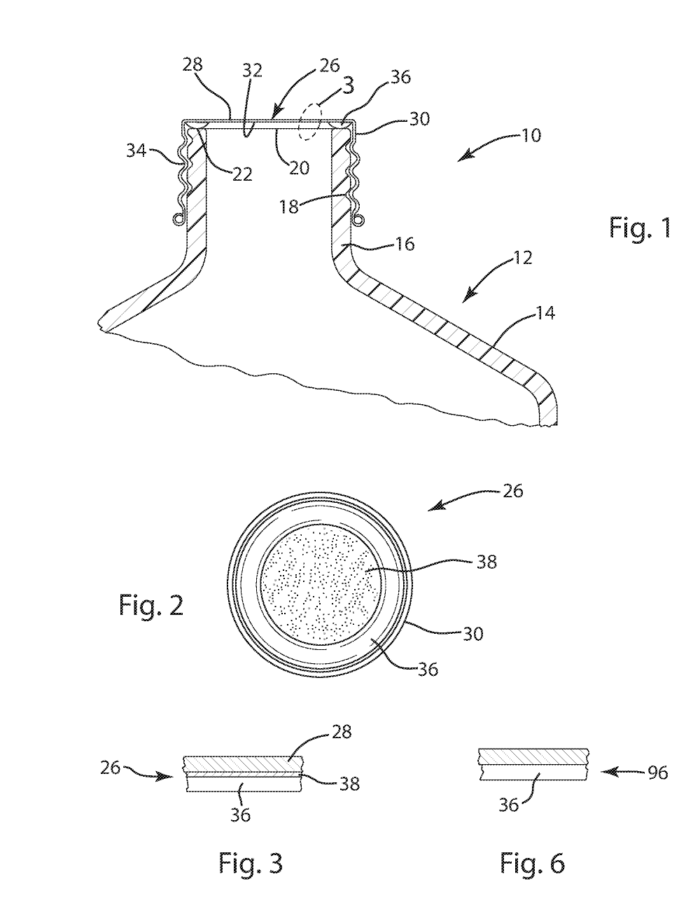

With reference now to the drawings in which like numerals represent like elements throughout the views, there is disclosed in FIG. 1 a pharmaceutical container 10 according to a first embodiment of the present invention. In FIG. 1, only a top portion of a gas-tight (and hence liquid-tight) pharmaceutical bottle 12 of pharmaceutical container 10 is schematically shown. Pharmaceutical bottle 12 is made of a suitable material in order to prevent any transfer of oxygen (or other gases, or moisture) in the atmosphere or environment through the walls thereof, as the oxygen (or other gases or liquids) would potentially damage pharmaceuticals inside, such as oxygen-sensitive pharmaceuticals or the like in any form (tablets, capsules, coated tablets, etc.), and especially ultra-oxygen-sensitive levothyroxine sodium tablets, capsules or the like. In this embodiment, pharmaceutical bottle 12 is designed for use by pharmacists, and thus holds large quantities (e.g., 1000) of tablets, capsules, or the like. Bottle 12 is thus made of a suitable plastics material such as high density polyethylene or polypropylene which are well known in the art for preventing oxygen ingress or which is well-known for preventing oxygen ingress together with a suitable lining layer; but pharmaceutical bottle 12 could also be glass or other like materials which prevent gas or liquid transfer therethrough if desired.

Pharmaceutical bottle 12 is only partially shown, but is typical of such bottles known in the art and is thus only schematically depicted in FIG. 1. Pharmaceutical bottle 12 includes a main enclosure 14 (only partially shown) with an upper neck 16 extending vertically therefrom. Upper neck 16 includes external screw threads 18, and upper neck 16 forms a top opening 20 for main enclosure 14 by which the pharmaceuticals in bottle 12 can be accessed (dispensed, spooned, shaken or poured out) by the user. An upper rim 22 is presented at the topmost part of upper neck 16, with upper rim 22 forming a planar engagement surface about top opening 20.

In order to provide a gas-tight closing of top opening 20 of main enclosure 14, pharmaceutical bottle 12 additionally includes a metal screw-on closure cap 26 as additionally shown in FIGS. 2 and 3. Closure cap 26 is not of the child-resistant type in view of its intended use by pharmacists (or alternatively by those not desiring a child-resistant cap with a regular sized pharmaceutical bottle). Conveniently, closure cap 26 is made of a suitable metal such as aluminum or steel as desired. Closure cap 26 includes a circular top member 28 having a circumferential flange 30 extending downwardly from a periphery thereof so as to define an interior area 32 of top member 28. Flange 30 has internal screw threads 34 which mate with external screw threads 18 of upper neck 16 to secure closure cap 26 under pressure (torqueing of the closure cap) to planar engagement surface of upper rim 22 of main enclosure 14.

The gas-tight closing supplied by closure cap 26 is provided by an annular pliable gasket 36 which is held in place or secured to a peripheral portion of interior area 32 of top member 28. Gasket 36 preferably has an outer convex shape and an inner flat shape as shown, whereby the convex shape can be suitably compressed to provide the gas-tight seal against upper rim 22 while the flat shape maintains its shape as it is pressed against the flat inside surface of top member 28. Gasket 36 is suitably made of butyl, silicone, nitrile or fluoroelastomer (such as VITON), or other acceptable pliant materials as known in the art, and it is sized to fit snuggly inside of flange 30 in order to be elastically held in place or in order to be adhered in place to the peripheral portion of interior area 32. In order to protect the pharmaceuticals from the metal of closure cap 26, and vice versa, a layer of lacquer 38 (whose size is exaggerated in FIG. 3 in order to be easily seen) is applied to interior area 32 of top member 28 at least inside of gasket 36. In operation then, the torqueing threaded engagement of screw-on closure cap 26 with pharmaceutical bottle 10 under pressure causes pliable gasket 36 to be compressed and thus to sealingly engage upper rim 22 of pharmaceutical bottle 12 to provide a gas-tight seal for top opening 20.

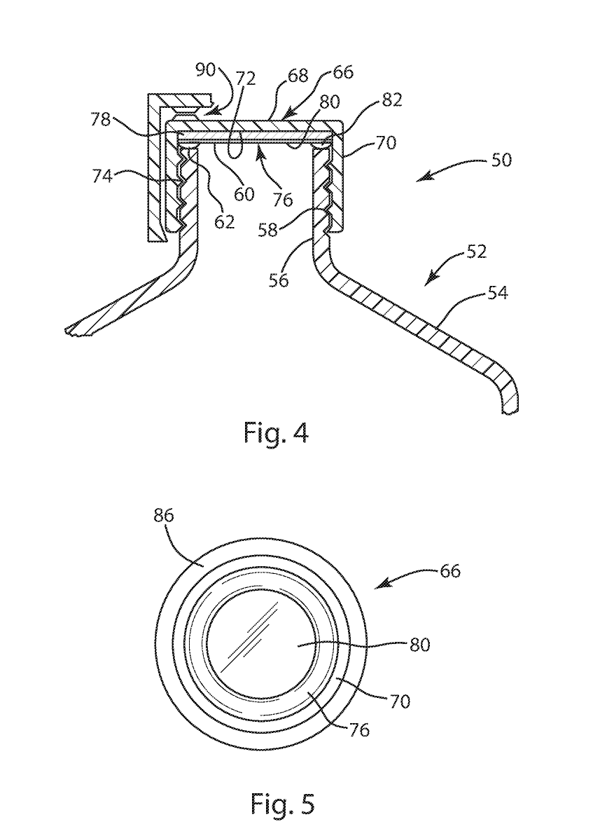

Depicted in FIGS. 4 and 5 is a pharmaceutical container 50 according to a second embodiment of the present invention. In FIG. 4, a top portion of a gas-tight pharmaceutical bottle 52 of pharmaceutical container 50 is schematically shown. Pharmaceutical bottle 52 in this embodiment is designed for consumer use to hold a single prescription (e.g., 10-100+, as desired). Bottle 52 is made of a suitable plastics material such as high density polyethylene or polypropylene, or a suitable plastics material coated with a suitable layer such as various resins or fluoride compounds, in order to prevent any transfer of oxygen in the atmosphere (or other gases or liquids) through the walls thereof in the same manner as pharmaceutical bottle 12. Pharmaceutical bottle 52 includes a main enclosure 54 with an upper neck 56 extending therefrom. Upper neck 56 includes external screw threads 58, and upper neck 56 forms a top opening 60 for main enclosure 54 by which the pharmaceuticals in bottle 52 can be accessed by the user. An upper rim 62 is presented at the topmost part of upper neck 56, with upper rim 62 forming a planar engagement surface about top opening 60.

In order to provide a gas-tight closing of top opening 60 of main enclosure 54, pharmaceutical bottle 52 additionally includes a plastic screw-on closure cap 66 as additionally shown in FIG. 5. Closure cap 66 is conveniently made of a suitable plastics material as known in the art such as polypropylene in order to be sufficiently strong or robust. Closure cap 66 includes a circular top member 68 having a circumferential flange 70 extending downwardly from a periphery thereof so as to define an interior area 72 of top member 68. Flange 70 has internal screw threads 74 which mate with external screw threads 58 of upper neck 56 to secure closure cap 66 under torqueing pressure to planar engagement surface upper rim 62 of main enclosure 54. Since bottle 52 is designed for consumer use, surrounding closure cap 66 to make closure cap 66 child-resistant as well known in the art is a floating outer cap 86 (only partially shown in FIG. 5). Outer cap 86 is held loosely to closure cap 66 by a foot 88 at the bottom thereof, as well known in the art. Finally, pressing down on outer cap 86 allows screwing and unscrewing of closure cap 66 by virtue of mating engagements 90 as also well known in the art.

The gas-tight closing supplied by closure cap 66 is provided by a circular inner liner or backer 76 which extends over interior area 72 of top member 68. This inner liner 76 includes an uppermost (wax cardboard, pulp, plastic, foam or the like) base layer 78 which is fittingly held in place or lightly secured to interior area 72 of top member 68 at the top of flange 70. Inner liner 76 also includes, below base layer 78 and hence facing the pharmaceutical in bottle 52, an aluminum foil layer 80, or any other suitable layer which provides an oxygen (or other gas and liquid) impermeable layer to prevent ingress of oxygen through the plastic material of top member 68 of closure cap 66. Finally, as shown, an annular pliable gasket is secured, either adhered or elastically held in place, to a peripheral area of foil layer 80 to provide a gas-tight seal in the same manner as gasket 36 discussed above. Foil layer 80 can either be adhered to base layer 78, or be free floating and instead adhered to gasket 82 which is held elastically in place similar to gasket 36. In operation then, threaded engagement of screw-on closure cap 66 (through pressing engagement on outer cap 86) with pharmaceutical bottle 50 under torqueing pressure causes pliable gasket 82 (together with base layer 78) to sealingly engage upper rim 62 of pharmaceutical bottle 52 to provide a gas-tight seal for top opening 60.

Depicted in FIG. 6 is a cross section (similar to that of FIG. 3) of a top portion of an alternative closure cap 96. Closure cap 96 is much like closure cap 26 or closure cap 66, and includes an annular pliable gasket 36 adjacent thereto, either secured thereto by adhering or the like or floating but held in place by a resilient fit with the circumferential flange of closure cap 96. It will be appreciated that closure cap 96 is made of a suitable material, either metal or plastic, which by itself is satisfactory for preventing oxygen or other egress. A suitable plastic material would be the same as that (and with a like coating, if any) of the bottle, or a suitable metal (not requiring a coating) such as aluminum.

With any of the constructions described above, it will be appreciated that the following is achieved with both disclosed embodiments:
a simple gas-resistant annual ring gasket is employed;
the simple ring gasket can be used with child-resistant as well as non child resistance containers;
the complications of heat sealing a foil laminate are avoided;
no re-torqueing of the closure cap after cooling of a seal is needed;
the temperature of the pharmaceutical bottle contents is not raised when the seal is provided;
the use of a pliant annular gasket helps to provide a good and reliable seal, even where there are some minor surface irregularities on the upper rim around the open top;
the opening and closing of the bottle using the closure cap is simple;
re-sealing of the main closure and then re-opening at the next use is easy and reliable, requires little effort, and can be easily accomplished by most users;
the closure cap can be used on a variety of pharmaceutical bottles, including glass as well as plastic; and
the gasket remains in the closure cap at all times, so a new seal is always reliably provided after each use.

It is an advantage of the present invention that the use of the rubber gasket eliminates the need for the induction heating and sealing of the prior art glue/seal provided on the foil disk, which prior art foil disc was adhered to the lips of the mouth of the container and formed a seal therewith. However, it will be appreciated that the peeling of the prior art foil disc also acted as a tamper-evident feature, which tamper-evidence is not part of the present invention since the foil disc remains in the closure cap to effect a good barrier together with the gasket each time the cap is re-applied. Thus, it is contemplated that a different tamper-evident seal would be employed with the present invention, such as a shrink wrap around the cap or the like as well known in the art.

The present invention has been described as being applicable to pharmaceutical containers or the like. However, it will be appreciated that the invention would also be applicable to other products requiring sealing from the environment, such as nutritional products like rapidly-oxidized nuts or other foods.

While the present invention has been described with respect to exemplary embodiments thereof, it will be understood by those of ordinary skill in the art that variations and modifications can be effected within the scope and spirit of the invention.

I claim:

1. A gas-tight pharmaceutical container in combination with a pharmaceutical composition,
the gas tight pharmaceutical container comprising:
a pharmaceutical bottle including
a main enclosure,
an upper neck extending from said main enclosure and forming a top opening for said main enclosure, said upper neck including external screw threads, and
an upper rim of said upper neck forming a planar engagement surface of said upper neck about said top opening of said upper neck;
a metal screw-on closure cap which is used to seal said top opening of said bottle and to repeatedly re-seal said top opening after a removal of said closure cap, said closure cap including
a circular top member, and
a circumferential flange depending downwardly from said top member and defining an interior area of said top member, said flange having internal screw threads which mate with said external screw threads of said upper neck to secure said closure cap under pressure to said planar engagement surface of said upper neck of said main enclosure, and
an annular pliable gasket secured to a peripheral portion of said interior area of said top member;
whereby threaded engagement of said screw-on closure cap with said pharmaceutical bottle under pressure causes said pliable gasket to sealingly engage said upper rim of said pharmaceutical bottle to provide a gas-tight seal; and
the pharmaceutical composition comprising an ultra-oxygen-sensitive composition, disposed inside that pharmaceutical bottle.

2. A pharmaceutical container as claimed in claim 1, wherein said closure cap is aluminum.

3. A pharmaceutical container as claimed in claim 1, wherein said closure cap is steel, and further including a layer of lacquer applied to said interior area of said top member inside of said gasket.

4. A pharmaceutical container as claimed in claim 1, wherein a material of said pliable gasket is one of butyl, silicone, nitrile or fluoroelastomer.

5. A pharmaceutical container as claimed in claim 1, wherein said pharmaceutical bottle is made of a plastics material.

6. A pharmaceutical container as claimed in claim 1, further including a layer of lacquer applied to said interior area of said top member inside of said gasket.

7. A pharmaceutical container as claimed in claim 1, wherein said gasket is adhered to said interior area of said top member.

8. A pharmaceutical container as claimed in claim 1, wherein said gasket is resiliently held in place by said circumferential flange in a position immediately adjacent said interior area of said top member.

9. A gas-tight pharmaceutical container with pharmaceutical composition disposed therein comprising:
a pharmaceutical bottle and a pharmaceutical composition,
the pharmaceutical bottle including:
a main enclosure,
an upper neck extending from said main enclosure and forming a top opening for said main enclosure, said upper neck including external screw threads, and
an upper rim of said upper neck forming a planar engagement surface of said upper neck about said top opening of said upper neck;
a screw-on closure cap which is used to seal said top opening of said bottle and to repeatedly re-seal said top opening after a removal of said closure cap, said closure cap including
a circular top member, and
a circumferential flange depending downwardly from said top member and defining an interior area of said top member, said flange having internal screw threads which mate with said external screws of said upper neck to secure said closure cap under pressure to said planar engagement surface of said upper neck of said main enclosure; and
a circular inner liner which extends over said interior area of said top member of said closure cap, said inner liner including
a base layer which is secured to said interior area of said top member at the top of said flange,
a foil layer adjacent to said base layer, and
an annular pliable gasket secured to a peripheral area of said foil layer;
whereby threaded engagement of said screw-on closure cap with said pharmaceutical bottle under pressure causes said pliable gasket to sealingly engage said upper rim of said pharmaceutical bottle to provide a gas-tight seal; and
the pharmaceutical composition comprises an ultra-oxygen-sensitive composition, the pharmaceutical composition disposed in the pharmaceutical bottle.

10. A pharmaceutical container as claimed in claim 6, wherein said foil layer is adhered to said base layer.

11. The pharmaceutical container as claimed in claim 9, wherein a material of said pliable gasket is one of butyl, silicone, nitrile or fluoroelastomer.

12. The pharmaceutical container as claimed in claim 9, wherein said pharmaceutical bottle is made of a plastics material, and wherein said closure cap is made of a plastic material.

13. The pharmaceutical container as claimed in claim 9, wherein said gasket is resiliently held in place by said circumferential flange in a position immediately adjacent said foil layer.

14. The pharmaceutical container as claimed in claim 9, wherein said gasket is adhered to said foil layer.

15. The pharmaceutical container as claimed in claim 9, wherein said base layer is made of wax cardboard.

16. The pharmaceutical container as claimed in claim 9, wherein said base layer is made of a pulp material.

17. The pharmaceutical container as claimed in claim 9, wherein said closure cap is child-resistant and further includes an outer cap about said top member and flange.

18. The gas-tight pharmaceutical container in combination with a pharmaceutical composition of claim 1, wherein the ultra-oxygen-sensitive composition is levothyroxine sodium.

19. The gas-tight pharmaceutical container in combination with a pharmaceutical composition of claim 18, wherein the levothyroxine sodium is in a form selected from the group consisting of tablets and capsules.

20. The pharmaceutical container of claim 9, wherein the ultra-oxygen-sensitive composition is levothyroxine sodium.
